(19) United States
(12) Patent Application Publication
(10) Pub. No.: US 2025/0316808 A1
(43) Pub. Date: Oct. 9, 2025

(54) BATTERY COMPRISING INSULATED CONNECTING STRIP

(71) Applicant: LITRONIK Batterietechnologie GmbH, Pirna (DE)

(72) Inventors: Marc Robert HAUER, Uster (CH); Christian WAURISCH, Dresden (DE)

(73) Assignee: LITRONIK Batterietechnologie GmbH, Pirna (DE)

(21) Appl. No.: 18/860,883

(22) PCT Filed: May 9, 2023
(86) PCT No.: PCT/EP2023/062175

§ 371 Date: Oct. 28, 2024

(30) Foreign Application Priority Data
May 10, 2022 (EP) 22172579.9

(51) Int. Cl. H01M 50/178; A61N 1/378; H01M 4/38; H01M 50/105; H01M 50/186; H01M 50/193; H01M 50/474; H01M 50/486; H01M 50/519

(52) U.S. Cl. CPC H01M 50/178; H01M 4/382; H01M 50/105; H01M 50/186; H01M 50/193; H01M 50/474; H01M 50/486; H01M 50/519; A61N 1/378; H01M 2220/30

(57) ABSTRACT

An electrochemical cell, including: a first electrode, a second electrode, a first connecting conductor, a second connecting conductor, a cell encapsulation, the first electrode and the second electrode being arranged inside the cell encapsulation and insulated with respect to the surrounding area, preferably in a fluid-tight manner, by the cell encapsulation, and the first connecting conductor and the second connecting conductor protruding from the cell encapsulation. It is provided that the first connecting conductor and the second connecting conductor are each, at least in sections, insulated with respect to the surrounding area, preferably in a fluid-tight manner, by an insulation, the insulated regions of the first connecting conductor and of the second connecting conductor protruding from the cell encapsulation, and a respective terminal section of the first connecting conductor and of the second connecting conductor not being covered by the insulation.

### CROSS-REFERENCE TO RELATED APPLICATIONS

This application is the United States National Phase under 35 U.S.C. § 371 of PCT International Patent Application No. PCT/EP2023/062175, filed on May 9, 2023, which claims the benefit of European Patent Application No. 22172579.9, filed on May 10, 2022, the disclosures of which are hereby incorporated by reference herein in their entireties.

### TECHNICAL FIELD

The present invention relates to an electrochemical cell, to an assembly, and to a medical device.

### BACKGROUND

In an effort to create very small, compact and low-cost wearables or implantable systems, a hermetic housing is dispensed with these days. So as to protect the electronic system of the wearable or of the implantable system against moisture, the electronic system is encapsulated or embedded in a plastic material. Advantageously, what are known as pouch cells can be used as the energy source for these applications, in which the components of the electrochemical cell (electrodes, current collectors, electrolyte, separator, and the like) are enclosed by a pouch instead of by a traditional battery housing. Such a pouch is typically formed of a multi-layer laminate, in which various layers (for example, made of aluminum, nylon, and propylene) are adhesively bonded to one another. Due to the temperature sensitivity of some batteries, the material selection for the encapsulation of batteries is limited since relatively high temperatures (>80° C.) are required for most materials used for encapsulation or embedding. Such high temperatures, however, cannot be used for the majority of batteries (primary or secondary cell).

Typical batteries, such as the aforementioned pouch cells, typically utilize non-insulated connecting strips or collectors, for example, in the form of wires or metal tabs or ribbons.

The region between the encapsulated electronic system and the housing of the battery, however, is not protected against environmental impact.

To protect this region, it would be possible to use an external housing, instead of an encapsulation, for the electronic system or the wearable or implanted system. This would bring with it the drawbacks of a not-so-compact design and higher manufacturing costs.

As an alternative, materials could be used that allow encapsulation at lower temperatures. Some of these materials, such as epoxy resins, however, cure only very slowly at low temperatures. This limits the throughput during production. Other materials use photoinitiators. Due to the complex geometries, however, uniform exposure is critical. Many of the materials furthermore inherently contain ionic components, which may result in the formation of dendrites, causing short circuits.

Based on this background, it is an objective of the present invention to provide a battery design that enables a secure connection of the battery to electronic system and a reliable and simple encapsulation of the electronic system, which also offers optimal protection against environmental conditions, and in particular moisture.

The present disclosure is directed toward overcoming one or more of the above-mentioned problems, though not necessarily limited to embodiments that do.

### SUMMARY

At least this object is achieved by an electrochemical cell having the features of claim 1, by an assembly having the features of claim 11, and by a device having the features of claim 15. Advantageous embodiments thereof are described in the corresponding dependent claim and in the following description.

According to claim 1, an electrochemical cell is provided. The electrochemical cell according to the present invention comprises:
a first electrode having a first polarity, the first electrode comprising a first current collector and a first electrode body comprising a first electrode material, and the first electrode body being electrically conductively connected to the first current collector;
a second electrode having a second polarity, the second electrode comprising a second current collector and a second electrode body comprising a second electrode material, and the second electrode body being electrically conductively connected to the second current collector;
a first connecting conductor, which is electrically conductively connected to the first current collector;
a second connecting conductor, which is electrically conductively connected to the second current collector; and
a cell encapsulation, the first electrode and the second electrode being arranged inside the cell encapsulation and insulated with respect to the surrounding area, preferably in a fluid-tight manner, by the cell encapsulation, and the first connecting conductor and the second connecting conductor protruding from the cell encapsulation.

According to the present invention, it is in particular provided that the first connecting conductor and the second connecting conductor are each, at least in sections, insulated with respect to the surrounding area, preferably in a fluid-tight manner, by an insulation, wherein the insulated regions of the first connecting conductor and of the second connecting conductor protrude from the cell encapsulation, and a respective terminal section of the first connecting conductor and of the second connecting conductor are not covered by the insulation.

The present invention is in particular based on the idea of localizing the encapsulation process of electronic system, for example, for a wearable or an implantable system, and of configuring the connecting conductors of the electrochemical cell in such a way that the hot encapsulation of the electronic system may be carried out away from the temperature-sensitive components of the battery, for example, by way of an appropriate length of the connecting conductors. At the same time, the connecting conductor is at least partially protected against environmental impact by the insulation according to the present invention, and in particular against liquids.

The electrochemical cell according to the present invention may be configured both as a primary cell and a secondary cell. The design of the electrochemical cell according to the present invention furthermore allows the use of all known anode materials in combination with corresponding cathode materials.

According to one embodiment of the electrochemical cell according to the present invention, the insulation of the first connecting conductor and/or of the second connecting conductor comprises or substantially consists of a plastic material. According to one embodiment, this plastic material is a thermoplastic material, and preferably a liquid crystal polymer.

Within the meaning of the present invention, the term “liquid crystal polymer” (or LCP) is used in the meaning known to and commonly used by a person skilled in the art. A “liquid crystal polymer” refers in particular to an aromatic polymer, which has highly ordered or crystalline regions in the molten state or in solution. Non-limiting examples include aromatic polyamides such as aramid (Kevlar) and aromatic polyesters of hydroxybenzoic acid, such as a polycondensate of 4-hydroxybenzoic acid and 6-hydroxynaphthalene-2-carboxylic acid (Vectran).

According to a further embodiment of the electrochemical cell according to the present invention, the cell encapsulation is designed in the form of a pouch, the pouch being in particular designed to be fluid-tight. The pouch may be formed of a multi-layer laminate, comprising multiple layers made of thermoplastic materials such as polypropylene, polyamide and/or a liquid crystal polymer and/or one or more metal foils, for example, made of aluminum, which advantageously may be integrally joined to one another, in particular by adhesive bonding. Instead of the metal foil, a metallized plastic film may also be used. As an alternative, the pouch may be substantially made of one material, in particular a thermoplastic material, and preferably a liquid crystal polymer.

According to a further embodiment of the electrochemical cell according to the present invention, the cell encapsulation comprises or substantially consists of a plastic material. According to one embodiment, this plastic material is a thermoplastic material, and preferably a liquid crystal polymer.

According to a further embodiment of the electrochemical cell according to the present invention, the cell encapsulation and the insulation of the first and second connecting conductors comprise or substantially consist of a liquid crystal polymer. Differing liquid crystal polymers may be used for the cell encapsulation and the insulations of the first and second connecting conductors. Advantageously, however, at least similar liquid crystal polymers are used for the insulations and the cell encapsulation, that is, having similar properties, such as the melting temperature. Preferably, the same liquid crystal polymer is used for the insulation and the cell encapsulation. Liquid crystal polymers in particular have the advantage of having very low permeability to water and gases. Since the liquid crystal polymers are a thermoplastic material, the interfaces may melt during the thermal processes, and the interface effects are considerably reduced.

According to a further embodiment of the electrochemical cell according to the present invention, the first current collector is formed by a metallization on an insulating plastic material, in particular a thermoplastic material, and preferably a liquid crystal polymer.

According to a further embodiment of the electrochemical cell according to the present invention, the second current collector is formed by a metallization on an insulating plastic material, in particular a thermoplastic material, and preferably a liquid crystal polymer.

According to a further embodiment of the electrochemical cell according to the present invention, the first current collector and the second current collector are each formed by a metallization on an insulating plastic material, in particular a thermoplastic material, and preferably a liquid crystal polymer.

According to a further embodiment of the electrochemical cell according to the present invention, the first current collector and the first connecting conductor are formed by a first conductor track of a first printed circuit board, wherein the first printed circuit board comprises an electrically insulating substrate, and the insulation of the first connecting conductor is at least partially formed by the substrate of the first printed circuit board. According to one embodiment, the electrically insulating substrate of the first printed circuit board comprises or substantially consists of a thermoplastic material, and preferably a liquid crystal polymer.

According to a further embodiment of the electrochemical cell according to the present invention, the second current collector and the second connecting conductor are formed by a second conductor track of a first printed circuit board, wherein the first conductor track and the second conductor track are electrically insulated from one another, and the insulation of the second connecting conductor is at least partially formed by the first substrate of the first printed circuit board.

According to an alternative embodiment of the electrochemical cell according to the present invention, the first current collector and the first connecting conductor are formed by a first conductor track of a first printed circuit board, wherein the first printed circuit board comprises an electrically insulating substrate, and the insulation of the first connecting conductor is at least partially formed by the substrate of the first printed circuit board, and the second current collector and the second connecting conductor are formed by a conductor track of a second printed circuit board, wherein the second printed circuit board comprises an electrically insulating substrate, and the insulation of the second connecting conductor is at least partially formed by the substrate of the second printed circuit board. According to one embodiment, the electrically insulating substrate of the second printed circuit board comprises or substantially consists of a thermoplastic material, and preferably a liquid crystal polymer. The electrically insulating substrate of the first printed circuit board and the electrically insulating substrate of the second printed circuit board preferably comprise the same liquid crystal polymer.

According to a further embodiment of the electrochemical cell according to the present invention, the cell encapsulation is formed in particular at least partially, and preferably substantially completely, by the substrate of the first printed circuit board. According to one embodiment, the substrate of the first printed circuit board comprises a first section and a second section, wherein the first section at least partially surrounds the first conductor track, and the second section at least partially surrounds the second conductor track, and the first section and the second section are integrally joined or firmly bonded to one another, in particular melted or welded together, preferably in a fluid-tight manner. In this way, advantageously both the cell encapsulation and the insulations of the first and second connecting conductors are formed by the substrate of the first printed circuit board.

For this purpose, for example, initially the first printed circuit board comprising the first and second conductor tracks may be provided, the first conductor track may be partially coated with the first electrode active material, and the second conductor track may be partially coated with the second electrode active material, whereby the first and second electrodes are formed. For the encapsulation, the first printed circuit board or the substrate of the first printed circuit board may then be folded about an axis in such a way that the first and second electrodes essentially oppose one another, and the first and second connecting conductors are offset from one another, wherein thereafter the first printed circuit board is integrally joined or firmly bonded with itself at the edges, in particular melted or welded together, so as to achieve an in particular fluid-tight encapsulation. In particular, the encapsulation may be designed in the form of a pouch. Thereafter, a respective portion of the first and of the second conductor track, which each form the first and second connecting conductor, may be freed of the material of the substrate, for example, by means of laser, so as to expose a portion of the first and second connecting conductors to the surrounding area. The electrochemical cell according to the present invention is, of course, only sealed or encapsulated by the first printed circuit board when all the necessary components, including at least one electrolyte and at least one separator, are arranged in the cell, or the electrochemical cell is initially only partially encapsulated so as to at least make it possible to add an electrolyte.

According to an alternative embodiment of the electrochemical cell according to the present invention, the cell encapsulation is formed in particular at least partially, and preferably substantially completely, by the substrate of the first printed circuit board and by the substrate of the second printed circuit board. According to one embodiment, the substrate of the first printed circuit board comprises a section that surrounds the first conductor track of the first printed circuit board, and the substrate of the second printed circuit board comprises a section that surrounds the conductor track of the second printed circuit board, wherein the aforementioned section of the first printed circuit board and the aforementioned section of the second printed circuit board are integrally joined or firmly bonded to one another, in particular melted or welded together, preferably in a fluid-tight manner. In this way, advantageously both the cell encapsulation and the insulations of the first and second connecting conductors are formed by the substrate of the first printed circuit board and the substrate of the second printed circuit board.

For this purpose, for example, the first electrode active material may be partially applied to the conductor track of the first printed circuit board, and the second electrode active material may be partially applied to the conductor track of the second printed circuit board, whereby the first and second electrodes are formed. Thereafter, the first and second printed circuit boards may be arranged with respect to one another in such a way that the first and second electrodes essentially oppose one another, and the first and second connecting conductors are offset from one another, wherein thereafter the first printed circuit board and the second printed circuit board are integrally joined or firmly bonded at the edges, in particular melted or welded together, so as to achieve an in particular fluid-tight encapsulation, wherein in particular the first and second printed circuit boards together may form a pouch. Thereafter, a respective portion of the conductor tracks of the first and second printed circuit boards, which each form the first and second connecting conductors, may be freed of the material of the substrate, for example, by means of laser, so as to expose a portion of the first and second connecting conductors to the surrounding area. Prior to the encapsulation, however, initially all the necessary components, including at least one electrolyte and at least one separator, are arranged in the cell. Initially, a partial encapsulation, which could at least allow electrolyte to be added, and a final encapsulation after the electrolyte has been added, would also be conceivable.

According to a further embodiment of the electrochemical cell according to the present invention, the first electrode body of the first electrode comprises an alkali metal as the electrode active material, preferably lithium, sodium or potassium, and the second electrode body of the second electrode comprises carbon monofluoride (CFx), manganese dioxide (MnO₂), iodine (I₂), silver vanadium oxide (SVO), copper silver vanadium oxide (CSVO), V₂O₂, TiS₂, CuO₂, Cu₂S, FeS, FeS₂, Ag₂O, Ag₂O₂, CuF, Ag₂CrO₄, CuO, Cu₂P₂O₇, Cu₄P₂O₉, Cu₅P₂O₁₀, Ag₂Cu₂P₂O₈, Ag₂Cu₃P₂O₉, copper vanadium oxide or a mixture thereof as the electrode active material. The second electrode body of the second electrode preferably comprises carbon monofluoride (CFx) or manganese dioxide (MnO₂) as the electrode active material. In the above-described embodiments, the electrochemical cell according to the present invention is in particular designed as a primary cell.

According to a further embodiment of the electrochemical cell according to the present invention, the first electrode body of the first electrode comprises an alkali metal as the electrode active material, preferably lithium, sodium or potassium, and the second electrode body of the second electrode comprises lithium cobalt oxide (LiCoO2), lithium nickel manganese cobalt oxides (mixed oxides of LiCoO2, LiNiO2 and LiMnO2), lithium nickel cobalt aluminum oxide (NCA), lithium manganese oxide (LiMn204), lithium iron phosphate (LiFePO4) or carbon, in particular graphite or hard carbon (or non-graphitizing carbon) as the electrode active material. In this embodiment, the electrochemical cell according to the present invention is in particular designed as a secondary cell.

According to a further embodiment, the electrochemical cell according to the present invention furthermore comprises an electrolyte, preferably a non-aqueous electrolyte. Suitable electrolytes include, without being limited thereto, non-aqueous, preferably aprotic, solvents, in particular esters, ethers and dialkyl carbonates, in particular tetrahydrofuran, methyl acetate, diglyme (bis(2-methoxyethyl)ether), triglyme (tris(2-methoxyethyl)ether), tetraglyme (tetra(2-methoxyethyl)ether), 1,2-dimethoxyethane, 1,2-diethoxyethane, 1-ethoxy-2-methoxyethane, dimethyl carbonate, diethyl carbonate, dipropyl carbonate, ethyl methyl carbonate, methyl propyl carbonate, ethyl propyl carbonate or a mixture thereof, or cyclic carbonates, cyclic esters, cyclic amides, in particular propylene carbonate, ethylene carbonate, butylene carbonate, γ-butyrolactone, N-methylpyrrolidinone or a mixture thereof. Suitable solvents also encompass polar non-aqueous solvents, such as acetonitrile, dimethyl sulfoxide, dimethyl formamide, dimethyl acetamide or a mixture thereof. The electrolyte can be present in liquid form or in the form of a gel, for example, by admixing a gelling agent, such as silicic acid.

According to a further embodiment, the electrolyte is designed as a solid electrolyte, for example, lithium iodide (LiI).

According to a further embodiment, the electrolyte comprises at least one conducting salt. In one embodiment, in which the first electrode body comprises an alkali metal as the electrode active material, the conducting salt is preferably an inorganic salt of the alkali metal of the first electrode (or anode). Suitable anions include, without being limited thereto, PF₆⁻, BF₄⁻, AsF₆⁻, SbF₆⁻, ClO₄⁻, O₂⁻, AlCl₄⁻, GaCl₄⁻, SCN⁻, SO₃(C₆F₅)⁻, C(SO₂CF₃)₃⁻, N(SO₂CF₃)₂⁻ and SO₃CF₃. In one embodiment, the conducting salt is present in a concentration in the range of 0.5 to 2.0 mol*1⁻¹, and preferably in the range of 0.8 to 1.5, 2.0 mol*1⁻¹.

According to a further embodiment, the electrochemical cell according to the present invention furthermore comprises a separator, which is arranged between the first electrode and the second electrode. Suitable separators include, without be limited to, non-metallic materials, in particular polymers, such as polyethylene or polypropylene, polyamides, such as nylon, or fluoropolymers, such as an ethylene-tetrafluoroethylene (ETFE) copolymer or polytetrafluoroethylene (PTFE). The separator is preferably designed in the form of a membrane and is advantageously wetted or saturated with a suitable electrolyte, in particular with an electrolyte according to the above-described embodiments, before the electrochemical cell according to the present invention is assembled.

According to claim 11, an assembly is provided. The assembly comprises an electrochemical cell according to claim 1 or one of the above-described embodiments thereof, and an electronic module, wherein the electronic module comprises a module substrate, a module conductor structure, one or more electronic module components and a module encapsulation, wherein the one or more electronic module components are arranged on the module substrate and electrically conductively connected to the module conductor structure, and wherein the first connecting conductor and the second connecting conductor of the electrochemical cell are electrically conductively connected, in particular joined, to the module conductor structure.

According to one embodiment of the assembly according to the present invention, a module encapsulation is at least partially formed by the module substrate.

According to a further embodiment of the assembly according to the present invention, the module substrate comprises a section that surrounds the module conductor structure and/or the one or more electronic module components, wherein the electronic module furthermore comprises a cover layer, which is arranged on the one or more electronic module components and/or the module conductor structure, and the cover layer is integrally joined or firmly bonded, in particular melted or welded, to the aforementioned section of the module substrate, in particular in a fluid-tight manner, and wherein the module encapsulation is at least partially, and preferably substantially completely, formed by the module substrate and the cover layer.

According to a further embodiment of the assembly according to the present invention, the module substrate and/or the cover layer comprise or substantially consist of a thermoplastic polymer, preferably a liquid crystal polymer. The module substrate and the cover layer preferably comprise the same liquid crystal polymer.

According to a further embodiment of the assembly according to the present invention, the module substrate and the first printed circuit board of the electrochemical cell are formed by a shared or common substrate.

According to claim 15, a device is provided, wherein the device comprises an electrochemical cell according to claim 1 or one of the above-described embodiments thereof, or an assembly according to claim 11 or one of the embodiments thereof. According to one embodiment, the device is designed as a medical device, and preferably as an implantable medical device. According to an alternative embodiment, the device is designed as a wearable device, or a wearable.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further features and advantages of the present invention are described hereafter based on the description of the figures of exemplary embodiments. In the drawings:

FIG. 1 shows an electrochemical cell according to the known prior art;

FIG. 2 shows an assembly comprising an electronic module and an electrochemical cell connected thereto of the known prior art;

FIG. 3 shows an embodiment of the electrochemical cell according to the present invention;

FIG. 4 shows an assembly comprising the embodiment of the electrochemical cell according to the present invention from FIG. 3;

FIG. 5 shows an assembly comprising a further embodiment of the electrochemical cell according to the present invention;

FIG. 6 shows an assembly comprising a further embodiment of the electrochemical cell according to the present invention; and

FIG. 7 shows the embodiment of the electrochemical cell according to the present invention from FIG. 6.

### DETAILED DESCRIPTION

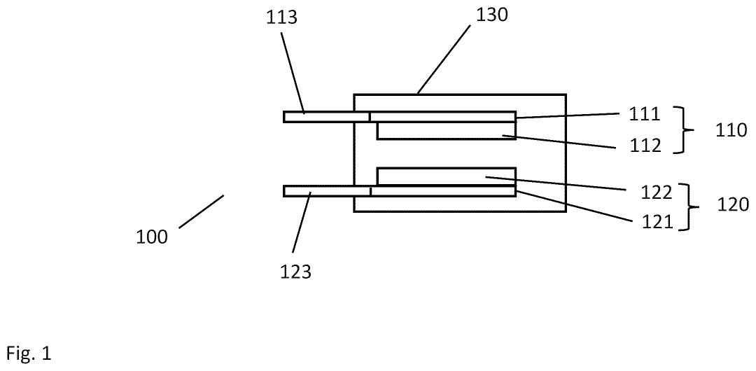

FIG. 1 shows an electrochemical cell 100, as it is known in the prior art, such as a lithium-ion cell. The cell comprises an anode 110, which comprises an anode body 112 including an anode material, such as lithium, which is applied to an anode collector or anode current collector 111. The electrochemical cell furthermore comprises a cathode 120 comprising a cathode body 122 including a cathode material (for example, CFx or MnO₂), which is applied to a cathode collector or current collector 121. The two electrodes 110, 120 are separated from the surrounding area by a suitable housing, for example, a welded composite film 130, which is designed as a pouch. For contacting other components (see below), the electrochemical cell 100 comprises a respective connecting conductor 113, 123 for the two electrodes, which are electrically conductively connected to the corresponding current collectors 111, 121 and which are guided out of the electrochemical cell through the housing or the composite film 130. In the case of regular batteries, the connecting conductors are typically designed as wires or metal tabs.

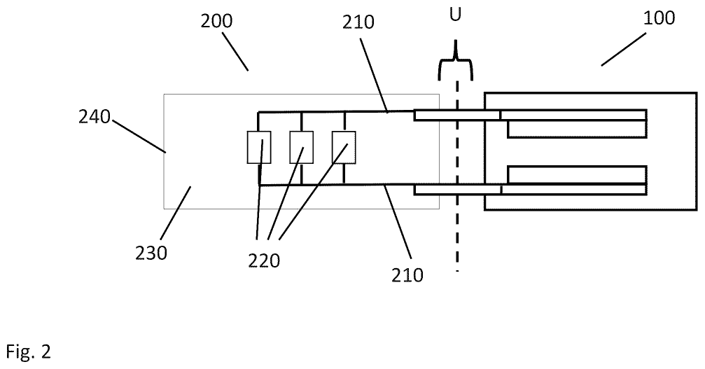

FIG. 2 shows an assembly comprising the above-described electrochemical cell 100, which is electrically conductively connected to an electronic module 200. The module may comprise a printed circuit board comprising a substrate 230 and a conductor structure 210, and additionally one or more electronic components 220, which are arranged on the printed circuit board and electrically conductively connected to the conductor structure 210. If the assembly is intended for use in compact wearables or implantable devices, the electronic module 200 is typically embedded in plastic material (such as LCP), instead of in a housing, as protection against environmental impact, for example, moisture. As was already mentioned above, typical batteries, such as the above-described electrochemical cell 100, use non-insulated connecting conductors/collectors 113, 123. The region U between the encapsulated electronic module 200 and the housing 130 of the electrochemical cell would thus not be protected. Thus, if only the end regions of the wires are also encapsulated, no sealed connection exists from the battery housing to the electronic system, since an unprotected region U still remains between the battery 100 and the electronic system 200.

To solve this problem, according to the present invention in particular an electrochemical cell 300, 400 is provided with an insulated 340, 440 connecting strip/conductor. As a result of a localization of the encapsulation process, these connecting strips/conductors 311, 321, 451, 452, 461 may be encapsulated, similarly to the electronic module 200, without exposing the electrochemical cell 300, 400 to temperatures.

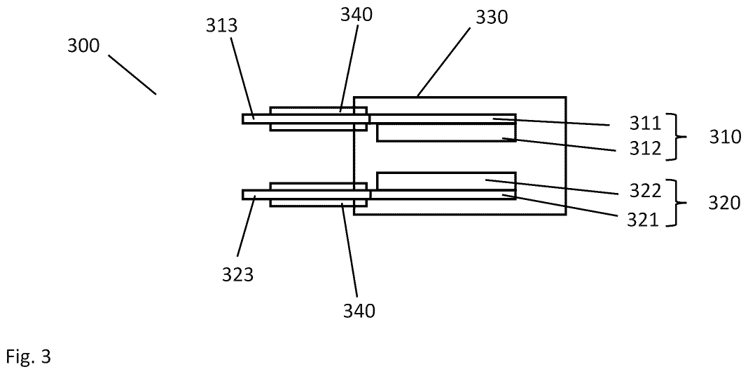

FIG. 3 shows an embodiment of the electrochemical cell 300 according to the present invention. In addition to typical components such as the anode 310, the cathode 320 (including the anode current collector 311 and the cathode current collector 321), the electrochemical cell 300 is in particular characterized by connecting conductors 313, 323 that have an insulation 340. The insulated 340 connecting strips/conductors 313, 323 are connected in the interior of the electrochemical cell 300 to the cathode and anode collectors or current collectors 311, 321. When the electrochemical cell 300 is being sealed, the connecting strips/conductors 311, 321 are guided to the outside through the cell encapsulation 330. The connecting strips/conductors 313, 323 comprise a terminal section that is at least partially free of the insulation 340. This section is used for electrically contacting a further component.

The cell encapsulation 330 may be formed by a laminate or a composite film, which is designed in the form of a bag (pouch). The laminate or the composite film may comprise multiple layers, in particular a metallic layer or a metallized layer, for example, made of or containing aluminum, which is coated on both sides with a plastic layer or coating, for example, made of nylon, polypropylene and/or LCP. For sealing the electrochemical cell 300 according to the present invention, the above-described components of the cell (electrodes 310, 320 including connected connecting conductors 313, 323) may be arranged on a first section of the above-described composite film, wherein at least one separator (not shown) is arranged between the electrodes 310, 320 in such a way that no direct physical and/or electrically conductive contact is present between the electrodes 310, 320. Thereafter, a second section of the composite film may be folded along a folding edge onto the first section, and the two edges of the first and second section, which are perpendicular to the folding axis, may be integrally joined or firmly bonded, for example, by adhesive bonding or welding. In this way, a pouch that is open toward one side may be produced from the composite film, wherein an electrolyte may be added through this opening. Furthermore, the connecting conductors 313, 323 may be guided via this opening out of the cell encapsulation 330 or out of the cell 300. Thereafter, the opening can be closed. The at least one separator may already be saturated with an electrolyte before being arranged between the electrodes 310, 320. As an alternative, the electrolyte may be present in the form of a gel and be applied to an electrode 310, 320 and/or the separator, before the latter are arranged on the composite film.

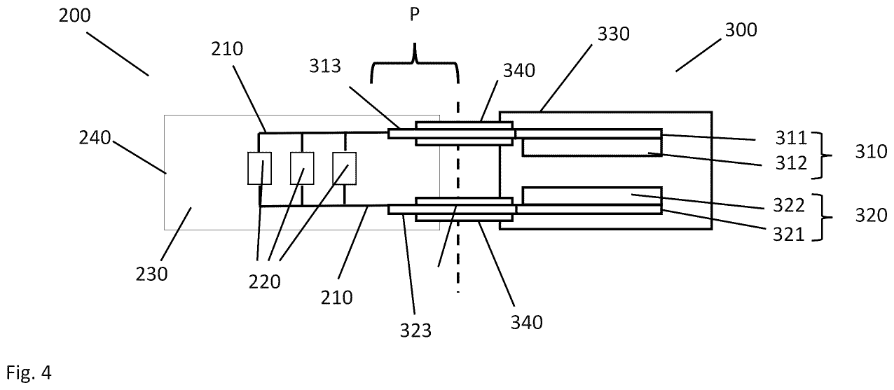

FIG. 4 shows an assembly comprising an electronic module 200 (comparable to the electronic module shown in FIG. 2). The above-described terminal sections of the connecting strips/conductors 313, 323 contact the module conductor structure 210 of the electronic module 200.

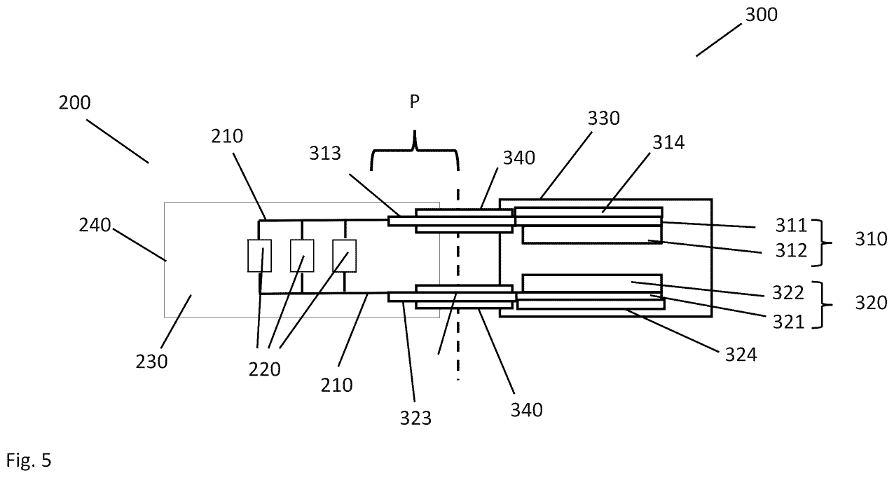

FIG. 5 shows an alternative embodiment, according to which the collector films or current collectors 311, 321 of the electrodes 310, 320 are made of conductive metallizations on the insulation material 314, 324. FIG. 5 in each case schematically shows the metallization 311, 321 on the respective insulation material 314, 324, which together form the respective current collector.

If the insulation 340 of the connecting strips/conductors 311, 321 is made of LCP (as in FIGS. 3 and 4), or if the collector films or current collectors are made of metallized/conductive LCP 311, 314, 321, 324 (as in FIG. 5), and if the electronic module 200 is likewise encapsulated with this thermoplastic, the connecting strips/conductors 311, 321 may be directly connected (for example, welded) or encapsulated directly with the electronic module 200. Advantageously, such an encapsulation 230 does not have any interfaces, whereby the diffusion, for example, of a liquid, is considerably reduced.

As another embodiment, an LCP substrate 450, 460 comprising a partial metallization 451, 452, 461 may be used as an insulated connecting strip 413, 423, pick-up or conductor electrode 411, 421 (substrate of the active mass of the electrodes), and housing or cell encapsulation 430 of the battery.

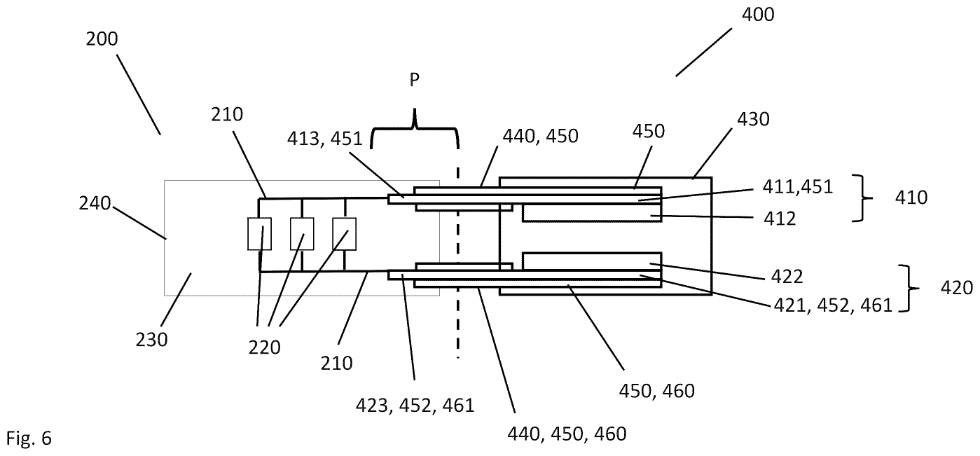

FIG. 6 shows such an embodiment in detail. A respective electrode is, or both electrodes 410, 420 are, essentially formed by a printed circuit board comprising an insulated substrate 450, for example, made of LCP, and one or more conductor tracks 451, 452. An electrode body 412, 422 is applied to a section of one of the conductor tracks 451, 452, which thereby forms the current collector 411, 421, wherein a further section of the conductor track 451, 452, which is not coated with the electrode body, forms the connecting conductor 413, 423. The two electrodes 410, 420, this being the anode and the cathode, may be formed on a shared or common substrate 450 with two conductor tracks 451, 452. As an alternative, an electrode 410 may be formed on a substrate 450 with one conductor track 451, and the other electrode 420 may be formed on another substrate 460 with another conductor track 461. In both instances, the substrate 450, 460 of the one or other printed circuit board forms a part of the insulation 440 of the connecting conductor.

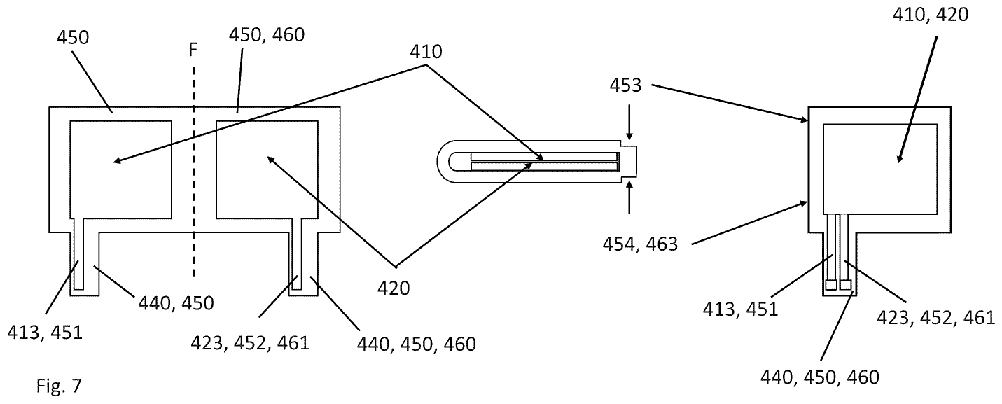

Furthermore, the one printed circuit board or both printed circuit boards may advantageously be used for the encapsulation 430 of the electrochemical cell 400. This is shown schematically in FIG. 7. In one variant, the two electrodes 410, 420 are formed on a printed circuit board or substrate 450. Thereafter, the printed circuit board or substrate is folded along an edge F so that the electrodes 410, 420 substantially congruently oppose one another, and the sections of the two conductor tracks 451, 452, which form the connecting conductors 413, 423, oppose one another in an offset manner, wherein, before or after folding, at least one separator is arranged between the electrodes 410, 420 in such a way that no direct physical and/or electrically conductive contact is present between the electrodes 410, 420. Thereafter, the substrate is welded onto itself via the sections 453, 454 of the substrate that extend around the electrode, and thereby an encapsulation 430 of the electrochemical cell 400 is achieved.

In an alternative variant, an electrode 410 is formed on a printed circuit board or substrate 450, as described above, and the other electrode 420 is formed on another printed circuit board or substrate 460. For the encapsulation, the two printed circuit boards or substrates 450, 460 are arranged in such a way that the electrodes 410, 420 substantially congruently oppose one another, and the sections of the conductor tracks 451, 461 which form the connecting conductors 413, 423 are offset from one another, wherein here as well, before the encapsulation, at least one separator is arranged between the electrodes 410, 420 in such a way that no direct physical and/or electrically conductive contact is present between the electrodes 410, 420. Thereafter, the two substrates 450, 460 are welded together via substrate sections 453, 463 that circumferentially extend around the respective electrodes 410, 420.

In the two above-described variants, an electrolyte can additionally be added to the electrochemical cell 400 prior to the encapsulation. As an alternative, the encapsulation 430 may initially only be carried out partially so as to form an opening for adding the electrolyte. This opening may then be completely closed after the electrolyte has been added.

In the implementation according to the present invention, it is thus possible to achieve a connection between the battery and the electronic system which has excellent electrical properties (low connection resistance, very high insulation) and very low diffusion (LCP may be welded together interface-free, the diffusion along interfaces is 100× to 1000× faster). At the same time, the temperature load of the battery may be avoided.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An electrochemical cell, comprising:
a first electrode having a first polarity, the first electrode comprising a first current collector and a first electrode body comprising a first electrode material, and the first electrode body being electrically conductively connected to the first current collector;
a second electrode having a second polarity, the second electrode comprising a second current collector and a second electrode body comprising a second electrode material, and the second electrode body being electrically conductively connected to the second current collector;
a first connecting conductor, which is electrically conductively connected to the first current collector;
a second connecting conductor, which is electrically conductively connected to the second current collector; and
a cell encapsulation, the first electrode and the second electrode being arranged inside the cell encapsulation and insulated with respect to the surrounding area, preferably in a fluid-tight manner, by the cell encapsulation, and the first connecting conductor and the second connecting conductor protruding from the cell encapsulation, wherein:
the first connecting conductor and the second connecting conductor are each, at least in sections, insulated with respect to the surrounding area, preferably in a fluid-tight manner, by an insulation, the insulated regions of the first connecting conductor and of the second connecting conductor protruding from the cell encapsulation, and a respective terminal section of the first connecting conductor and of the second connecting conductor not being covered by the insulation.

2. The electrochemical cell according to claim 1, wherein:
the insulation of the first connecting conductor and/or of the second connecting conductor comprises or substantially consists of a plastic material, in particular a thermoplastic material, and preferably a liquid crystal polymer, and/or
the cell encapsulation comprises or substantially consists of a plastic material, in particular a thermoplastic material, and preferably a liquid crystal polymer.

3. The electrochemical cell according to claim 1, wherein:
the first current collector is formed by a metallization on an insulating plastic material, in particular a thermoplastic material, and preferably a liquid crystal polymer; and/or
the second current collector is formed by a metallization on an insulating plastic material, in particular a thermoplastic material, and preferably a liquid crystal polymer.

4. The electrochemical cell according to claim 1, wherein the first current collector and the first connecting conductor are formed by a first conductor track of a first printed circuit board, the first printed circuit board comprising an electrically insulating substrate, and the insulation of the first connecting conductor being at least partially formed by the substrate of the first printed circuit board.

5. The electrochemical cell according to claim 4, wherein:
the second current collector and the second connecting conductor are formed by a second conductor track of the first printed circuit board, the first conductor track and the second conductor track being electrically insulated from one another, and the insulation of the second connecting conductor being at least partially formed by the substrate of the first printed circuit board; or
the second current collector and the second connecting conductor are formed by a conductor track of a second printed circuit board, the second printed circuit board comprising an electrically insulating substrate, and the insulation of the second connecting conductor being at least partially formed by the substrate of the second printed circuit board.

6. The electrochemical cell according to claim 4, wherein:
the substrate of the first printed circuit board comprises or substantially consists of a thermoplastic material, and preferably a liquid crystal polymer; and/or
the substrate of the second printed circuit board comprises or substantially consists of a thermoplastic material, and preferably a liquid crystal polymer.

7. The electrochemical cell according to claim 4, wherein the cell encapsulation is formed by the substrate of the first printed circuit board and/or by the substrate of the second printed circuit board.

8. The electrochemical cell according to claim 7, wherein:
the substrate of the first printed circuit board comprises a first section and a second section, the first section at least partially surrounding the first conductor track, and the second section at least partially surrounding the second conductor track, and the first section and the second section being integrally joined to one another, in particular melted or welded together, preferably in a fluid-tight manner; or
the substrate of the first printed circuit board comprises a section that surrounds the first conductor track of the first printed circuit board, and the substrate of the second printed circuit board comprises a section that surrounds the conductor track of the second printed circuit board, the aforementioned section of the first printed circuit board and the aforementioned section of the second printed circuit board being integrally joined to one another, in particular melted or welded together, preferably in a fluid-tight manner.

9. The electrochemical cell according to claim 1, wherein the first electrode body of the first electrode comprises an alkali metal as an electrode active material, preferably lithium, sodium or potassium.

10. The electrochemical cell according to claim 1, furthermore comprising:
an electrolyte, preferably a non-aqueous electrolyte; and/or
a separator, the separator being arranged between the first electrode and the second electrode.

11. An assembly, comprising:
the electrochemical cell according to claim 1; and
an electronic module, the electronic module comprising a module substrate, a module conductor structure, one or more electronic module components and a module encapsulation, the one or more electronic module components being arranged on the module substrate and electrically conductively connected to the module conductor structure,
the first connecting conductor and the second connecting conductor of the electrochemical cell being electrically conductively connected, in particular joined, to the module conductor structure.

12. The assembly according to claim 11, wherein the module encapsulation is at least partially formed by the module substrate.

13. The assembly according to claim 12, wherein the module substrate comprises a section that surrounds the module conductor structure and/or the one or more electronic module components, the electronic module furthermore comprising a cover layer, which is arranged on the one or more electronic module components and/or the module conductor structure, and the cover layer being integrally joined, in particular melted or welded, to the aforementioned section of the module substrate, in particular in a fluid-tight manner, and the module encapsulation being at least partially formed by the module substrate and the cover layer.

14. The assembly according to claim 11, wherein the module substrate and/or the cover layer comprise or substantially consist of a thermoplastic polymer, and preferably a liquid crystal polymer.

15. A device, in particular a medical device or an implantable medical device, or a portable device, comprising the electrochemical cell according to claim 1.

16. A device, in particular a medical device or an implantable medical device, or a portable device, comprising the assembly according to claim 11.
